(19) United States
(12) Patent Application Publication
(10) Pub. No.: US 2012/0066965 A1
(43) Pub. Date: Mar. 22, 2012

(54) CATALYST SYSTEMS FOR BIODIESEL PRODUCTION

(75) Inventors: Johannes Ruwwe, Niederkassel (DE); Martin Lichtenheldt, Koeln (DE); Wolfgang-Wilhelm Orlia, Toenisvorst (DE)

(73) Assignee: EVONIK DEGUSSA GmbH, Essen (DE)

(21) Appl. No.: 13/234,293

(22) Filed: Sep. 16, 2011

(30) Foreign Application Priority Data
Sep. 17, 2010 (DE) 10 2010 040 939.1

(51) Int. Cl. C10L 1/19; C07C 67/02

(52) U.S. Cl. 44/403; 554/167

(57) ABSTRACT

A method to transesterify triglycerides in the presence of a catalyst system comprising a transesterification catalyst selected from the group of the alkali metal and alkaline earth metal alkoxides and the alkali metal hydroxides, and at least one activator other than the transesterification catalyst, selected from the group comprising salt compounds, titanates and non-salt compounds having a density of at least 0.9 g/ml, is provided. Additionally provided is a method to prepare biodiesel by transesterification of a natural fat.

### CROSS-REFERENCE TO RELATED APPLICATIONS

This application claims priority to German Application No. 102010040939.1, filed Sep. 17, 2010, the disclosure of which is incorporated herein by reference in its entirety.

### FIELD OF THE INVENTION

The present invention relates to the use of a catalyst system comprising a transesterification catalyst selected from the group of the alkali metal and alkaline earth metal alkoxides and the alkali metal hydroxides, and at least one activator other than the transesterification catalyst, selected from the group comprising salt compounds, titanates or non-salt compounds having a density of at least 0.9 g/ml, for catalysis of transesterification reactions.

### BACKGROUND OF THE INVENTION

For some time, fatty acid alkyl esters of monohydric alcohols have found an important application in use as biodiesel, a substitute based on renewable raw materials for fossil diesel.

Biodiesel is generally produced by means of base-catalysed transesterification (The Biodiesel Handbook, G. Knothe, J. van Gerpen, J. Krahl, Ed. AOCS Press (2005); Biodiesel—The comprehensive handbook, M. Mittelbach, C. Remschmidt (2004); Bioresource Technology 2004, 92, 297; Applied Energy 2010, 87, 1083; Chimica Oggi/Chemistry today 2008, 26).

The most frequently used catalysts are sodium methoxide (NaOMe), sodium hydroxide (NaOH), potassium methoxide (KOMe) and potassium hydroxide (KOH). These catalysts are typically used as homogeneous catalysts dissolved in the monohydric alcohol used, for example methanol.

An alternative to this which has been described is that alkaline catalysts can be used in conjunction with phase transfer catalysts (WO 2007/111604). The phase transfer catalysts ensure that, compared to the use of the alkaline catalyst without phase transfer catalyst, the reaction is accelerated and a fuller conversion is achieved. A disadvantage of the process described is that the phase transfer catalysts are expensive and frequently corrosive because they contain chloride, bromide or other ions, to which the steel reactors in which biodiesel is typically produced are not resistant. In addition, there is a risk that small amounts of the phase transfer catalyst cannot be removed from the biodiesel by workup thereof.

Another way of modifying the reaction mixture is specified in publications DE 332506, DE 3415529, DE 102006044467 and DE 102007056703, which disclose that a portion of the amount of glycerol obtained in the transesterification process is recycled and added to the reaction mixture composed of triglyceride, monohydric alcohol and alkaline catalyst. This process allows the catalyst to be dispensed with; a disadvantage thereof is that addition of glycerol shifts the equilibrium of the transesterification reaction to the side of the reactants, and insufficient conversion is achieved under some circumstances.

There have additionally been descriptions of the possibility of improving the process for producing biodiesel by adding to the reaction mixture, after the transesterification, additives with which the phase separation to remove the glycerol released is accelerated.

Eur. J. Lipid Sci. Technol. 2008, 110, 347 describes the addition of water for this purpose. A disadvantage of this process is the fact that the addition of water can result in an unwanted hydrolysis reaction, which can reduce the biodiesel yield when the alkaline catalyst is not neutralized beforehand.

CN 101423773 describes the addition of calcium salts or magnesium salts to the reaction mixture after the transesterification, likewise with the aim of accelerating the phase separation. With some of the salts described, there may be problems with unwanted solids formation due to the poor solubility.

Both in the case of addition of water and in the case of addition of the calcium salts or magnesium salts, it is disadvantageous that the additive is added only after the reaction, which means additional apparatus complexity and time demands.

A further method of improving biodiesel production using homogeneous catalysts is the use of cosolvents, as described, for example, in Chemical Engineering Journal 2009, 146, 302; Energy&Fuels 2008, 22, 2702, or Biomass&Bioenergy 1996, 11, 43.

Although these cosolvents accelerate the reaction, they worsen or prevent the phase separation to remove the glycerol. Furthermore, the cosolvents have to be removed from the biodiesel and glycerol in a costly and inconvenient manner.

It was therefore an object of the present invention to provide a simplified process for transesterifying glycerides with monohydric alcohols, which brings about a faster and more complete reaction.

### DESCRIPTION OF THE PREFERRED EMBODIMENTS

This and other objects have been accomplished by the present invention, a first embodiment of which provides a method for preparation of a fatty acid alkyl ester, comprising:

transesterifying at least one of a mono-, di- or triglyceride with a monohydric alcohol in the presence of a catalyst system to obtain a phase separated product mixture comprising a fatty acid alkyl ester phase and a glycerol phase;

wherein the catalyst system comprises:

a transesterification catalyst selected from the group consisting of an alkali metal, an alkaline earth metal alkoxide and an alkali metal hydroxide; and

at least one activator, different from the transesterification catalyst, selected from the group consisting of a salt compound, a titanate and a non-salt compound having a density of at least 0.9 g/ml.

In a preferred embodiment, the invention provides a method for preparing a biodiesel, comprising:

transesterifying at least one natural fat with a monohydric alcohol in the presence of a catalyst system to obtain a phase separated product mixture comprising a biodiesel phase and a glycerol phase; and

separating the glycerol phase from the biodiesel phase to obtain the biodiesel;

wherein the catalyst system comprises:

a transesterification catalyst selected from the group consisting of an alkali metal, an alkaline earth metal alkoxide and an alkali metal hydroxide; and

at least one activator, different from the transesterification catalyst, selected from the group consisting of a salt compound, a titanate and a non-salt compound having a density of at least 0.9 g/ml.

It has been found that, surprisingly, multicomponent catalysts, i.e. mixtures of different catalysts, or conventional catalysts with suitable additions, either accelerate the transesterification reaction and/or improve the phase separation.

Accordingly, the present invention firstly provides for the use of a catalyst system comprising a transesterification catalyst selected from the group of the alkali metal and alkaline earth metal alkoxides and the alkali metal hydroxides, and at least one activator other than the transesterification catalyst, selected from the group comprising salt compounds, titanates and non-salt compounds having a density of at least 0.9 g/ml, for catalysis of transesterification reactions.

Compared to conventional catalysts which contain only one component, the multicomponent catalysts described have the advantage that the transesterification reaction and/or the phase separation of the glycerol released is accelerated, thus achieving a faster and more complete process and/or simplified biodiesel processing. The faster phase separation in particular constitutes a considerable advantage because the biodiesel production may thus additionally be rationalized. The catalyst systems used may bring about a faster and more complete phase separation of the glycerol released because the glycerol phase which forms has a higher density and/or a greater polarity.

When the catalyst system of this invention is used in the transesterification, costly and inconvenient later addition of additives is unnecessary.

The inventive catalyst system used has at least two components, the transesterification catalyst and at least one activator.

The transesterification catalyst is responsible for the actual transesterification and is selected from the group of the alkali metal and alkaline earth metal alkoxides and the alkali metal hydroxides. Preferred transesterification catalysts are sodium methoxide, potassium methoxide, sodium ethoxide, potassium ethoxide, sodium hydroxide or potassium hydroxide. Very particular preference is given to using sodium methoxide or potassium methoxide as transesterification catalysts.

Typically, the transesterification catalysts are present in solution, and they especially comprise alcoholic solutions, preferably methanolic or ethanolic solutions. Most preferably, the alcohol used corresponds to the alkoxide used. Thus, the transesterification catalyst used is especially sodium methoxide in methanol or potassium methoxide in methanol.

In addition, the catalyst system contains at least one activator other than the transesterification catalyst. Said activator may be selected from the group comprising salt compounds, titanates and non-salt compounds having a density of at least 0.9 g/ml.

Salt compounds in the context of the present invention are understood to mean compounds which have a cation and an anion. These are especially chlorides, bromides, fluorides, acetates, formates, phosphates, hydrogenphosphates, sulphates, hydrogensulphates, nitrates, carbonates, hydrogencarbonates, cyanides, cyanates, thiocyanates, borates, silicates, aluminates, alkoxides or hexacyanoferrates of sodium, potassium, magnesium, calcium, zinc or iron. The term “alkoxides” encompasses the corresponding methoxides, ethoxides, n-propoxides, isopropoxides, tert-butoxides or tert-pentoxides.

Preference may be given to using methoxides and ethoxides, very particular preference may be to using methoxides.

It may be likewise possible to use titanates, especially tetramethyl titanate Ti(OMe)₄, tetraethyl titanate Ti(OEt)₄ or tetraisopropyl titanate Ti(O-iso-Pr)₄.

Additionally suitable may be non-salt compounds having a density of at least 0.9 g/ml. The density is determined by methods commonly known to those skilled in the art, for example by means of the aerometer process (e.g. DIN EN ISO 3675) or pycnometer process.

The non-salt compounds may be organic compounds, preferably ethylene glycol, diethylene glycol, formamide, dimethylformamide, N-methylformamide, acetamide, dimethylacetamide, N-methylacetamide, N-ethylacetamide, propanamide, N-methylpropanamide, N-ethylpropanamide, N-methylpyrrolidone and/or dimethyl sulphoxide, most preferably dimethylformamide.

Among the activators mentioned, very particular preference may be given to using potassium methoxide, potassium formate, potassium phosphate or dimethylformamide. These activators are advantageous because they are inexpensive, readily available, and in the concentrations used, have good solubility in the glycerol phase which forms.

An essential feature of the catalyst system used may be that the transesterification catalyst and the activator are different from one another. This relates more particularly to the inventive embodiment in which both the transesterification catalyst and the activator are an alkali metal or alkaline earth metal alkoxide. When sodium methoxide is used as the transesterification catalyst, potassium methoxide may be used as the activator. Conversely, the use of sodium methoxide as the activator may be conceivable when potassium methoxide is used as the transesterification catalyst.

Very particularly preferred catalyst systems comprise sodium methoxide with potassium methoxide, sodium methoxide with potassium formate, sodium methoxide with dimethylformamide, potassium methoxide with potassium formate, and potassium methoxide with dimethylformamide.

The catalyst systems mentioned may be suitable for use in transesterification reactions, and there are no fundamental restrictions with regard to the type of transesterification reaction. A transesterification reaction in the context of the present invention is understood to mean a reaction in which a reactant ester and an alcohol are reacted with one another in the presence of the catalyst system, such that the alcohol reacts with the acid component of the reactant ester to give a correspondingly novel product ester, releasing the alcohol component of the reactant ester. Preference may be given to using the catalyst system for preparation of fatty acid alkyl esters by transesterification of mono-, di- or triglycerides. This converts mono-, di- or triglycerides to the corresponding fatty acid alkyl esters to simultaneously give free glycerol.

Accordingly, the present invention further provides a process for preparing fatty acid alkyl esters, comprising the transesterification of at least one mono-, di- or triglyceride in the presence of at least one monohydric alcohol, wherein a catalyst system comprising a transesterification catalyst selected from the group of the alkali metal and alkaline earth metal alkoxides and the alkali metal hydroxides, and at least one activator other than the transesterification catalyst, selected from the group comprising salt compounds, titanates and non-salt compounds having a density of at least 0.9 g/ml, is present.

The usable catalyst systems have been described above. Starting materials for the process according to the invention are mono-, di- and triglycerides of the general formula (I)

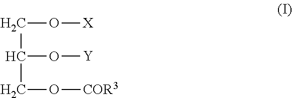

in which X═COR¹ or H, Y═COR² or H and R¹, R² and R³, which may be the same or different, are each aliphatic hydrocarbyl groups having 3 to 23 carbon atoms, where these groups may optionally be substituted by an OH group, or desired mixtures of such glycerides.

Thus, in glycerides of the formula (I), one or two fatty acid esters may be replaced by hydrogen. The fatty acid esters R¹CO—, R²CO— and R³CO— derive from fatty acids having 3 to 23 carbon atoms in the alkyl chain. R¹ and R² or R^{I}, R² and R³, in the above formula may be the same or different when the compounds are di- or triglycerides. The R¹, R² and R³ radicals may belong to the following groups:

a) alkyl radicals which may be branched but are preferably straight-chain and have 3 to 23, preferably 7 to 23, carbon atoms;

b) olefinically unsaturated aliphatic hydrocarbyl radicals which may be branched but are preferably straight-chain and have 3 to 23, preferably 11 to 21 and especially 15 to 21 carbon atoms, and which contain 1 to 6, preferably 1 to 3, double bonds which may be conjugated or isolated;

c) monohydroxy-substituted radicals of the a) and b) type, preferably olefinically unsaturated olefin radicals which have 1 to 3 double bonds, especially the radical of ricinoleic acid.

The acyl radicals R¹CO—, R²CO— and R³CO— of those glycerides which are suitable as starting materials for the process of the present invention may derive from the following groups of aliphatic carboxylic acids (fatty acids):

a) Alkanoic acids or the alkyl-branched, especially methyl-branched, derivatives thereof, which have 4 to 24 carbon atoms, for example butyric acid, valeric acid, caproic acid, heptanoic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachic acid, behenic acid, lignoceric acid, 2-methylbutanoic acid, isobutyric acid, isovaleric acid, pivalic acid, isocaproic acid, 2-ethylcaproic acid, the positionally isomeric methylcapric acids, methyllauric acids and methylstearic acids, 12-hexylstearic acid, isostearic acid or 3,3-dimethylstearic acid.

b) Alkenoic acids, alkadienoic acids, alkatrienoic acids, alkatetraenoic acids, alkapentaenoic acids and alkahexaenoic acids, and the alkyl-branched, especially methyl-branched, derivatives thereof, having 4 to 24 carbon atoms, for example crotonic acid, isocrotonic acid, caproleic acid, 3-lauroleic acid, myristoleic acid, palmitoleic acid, oleic acid, elaidic acid, erucic acid, brassidic acid, 2,4-decadienoic acid, linoleic acid, 11,14-eicosadienoic acid, eleostearic acid, linolenic acid, pseudoeleostearic acid, arachidonic acid, 4,8,12,15,18,21-tetracosahexaenoic acid or trans-2-methyl-2-butenoic acid.

c₁) Monohydroxyalkanoic acids having 4 to 24 carbon atoms, preferably having 12 to 24 carbon atoms, preferably unbranched, for example hydroxybutyric acid, hydroxyvaleric acid, hydroxycaproic acid, 2-hydroxydodecanoic acid, 2-hydroxytetradecanoic acid, 15-hydroxypentadecanoic acid, 16-hydroxyhexadecanoic acid, hydroxyoctadecanoic acid.

c₂) Also monohydroxyalkenoic acids having 4 to 24, preferably having 12 to 22 and especially having 16 to 22 carbon atoms (preferably unbranched) and having 1 to 6, preferably 1 to 3, ethylenic double bonds, and especially having one ethylenic double bond, for example ricinoleic acid or ricinelaidic acid.

Preferred starting materials for the process according to the invention may in particular be the natural fats, which are mixtures of predominantly triglycerides and small proportions of diglycerides and/or monoglycerides, these glycerides usually also in turn being mixtures and containing different types of fatty acid radicals within the abovementioned range, especially those having 8 or more carbon atoms. Examples include vegetable fats, such as olive oil, coconut fat, palm kernel fat, babassu oil, palm oil, palm kernel oil, peanut oil, rapeseed oil (corza oil), castor oil, sesame oil, sunflower oil, soya oil, hemp oil, poppy oil, avocado oil, cottonseed oil, wheatgerm oil, maize kernel oil, pumpkinseed oil, tobacco oil, grapeseed oil, jatropha oil, algae oil, karanja oil (oil of Pongamia pinnata), camelina oil (linseed dotter oil), cocoabutter, or else plant tallows, and also animal fats, such as bovine tallow, pork fat, chicken fat, bone fat, mutton tallow, japan tallow, whale oil and other fish oils, and also train oil. However, it may be equally also possible to use homogeneous tri-, di- and monoglycerides, whether they have been isolated from natural fats or obtained by a synthetic route. Examples here include: tributyrin, tricapronin, tricaprylin, tricaprinin, trilaurin, trimyristin, tripalmitin, tristearin, triolein, trielaidin, trilinolein, trilinolenin, monopalmitin, monostearin, monoolein, monocaprinin, monolaurin, monomyristin or mixed glycerides, for example palmitodistearin, distearoolein, dipalmitoolein or myristopalmitostearin.

Monohydric alcohols in the context of the present invention are understood to mean alcohols having only one OH group. Examples of monohydric alcohols are methanol, ethanol, n-propanol, isopropanol and n-butanol, isobutanol, sec-butanol or tert-butanol, and also branched or relatively long-chain, optionally likewise branched alcohols, for example amyl alcohol, tert-amyl alcohol, n-hexanol and/or 2-ethylhexanol. Preference is given to using methanol and ethanol. The alcohols mentioned can be used alone or in mixtures in the process according to the invention.

The concentration of the transesterification catalyst may be 0.001-20% by weight. This range includes all values and subvalues therebetween, preferably including 0.01-5% by weight and more preferably, including 0.1-2% by weight, based on the amount of mono-, di- or triglyceride used.

The amount of activator used may be 0.01-30% by weight. This range includes all values and subvalues therebetween, preferably including 0.1-20% by weight and most preferably 1-15% by weight, based on the amount of transesterification catalyst used.

The process according to the invention may be performed in all ways known to those skilled in the art. In the course of performance of the process according to the invention, the reaction mixture may preferably be stirred. The preferred intensive mixing of the reaction mixture may, however, also be achieved by other methods familiar to the person skilled in the art.

The reaction time may preferably be selected within the range from 1 to 120 minutes. This achieves conversions of at least 98%, preferably at least 99%. The conversion of the reaction may be based on the proportion of glycerides (=sum of tri-, di- and monoglycerides) still present: after the end or the stoppage of the reaction, based on the starting content of these components in the oil or fat used. The conversion may be determined by gas chromatography in a simple manner and is calculated from the contents of the alkyl esters divided by the sum of the contents of alkyl esters plus glycerides. The fatty acid alkyl esters obtainable by the process according to the invention may be used as biodiesel. According to the specification in DIN EN 14214, biodiesel may not contain more than 0.2% triglycerides according to test method EN 14105. In conventional transesterification processes which use equimolar amounts of NaOH but no further activators, conversions of the order of magnitude of >99.8% are achieved only after a prolonged period. An increase in the NaOH concentration to enhance the reaction rate in these conventional transesterification processes is undesirable since NaOH tends to hydrolyse mono-, di- or triglycerides or the corresponding alkyl esters to form the corresponding soaps, which firstly cause product losses and also have emulsifying action. Phase separation after the reaction has ended to separate the alkyl ester phase and glycerol phase is complicated or prevented as a result. Workup of the product is then possible only with difficulty.

The process according to the invention may be performed batchwise or continuously (for example in a tubular reactor, stirred tank, stirred tank cascade, or other processes known to those skilled in the art).

Preference may be given to establishing, in the process according to the invention, a molar ratio (monohydric alcohol: mono-, di-, triglyceride) in the range from 3:1 to 20:1. A ratio of 4:1 to 8:1 is very particularly preferred.

The catalyst system may preferably be used as a solution in the monohydric alcohol used, the actual transesterification catalyst being fully dissolved, while the activator may be present in completely or else only partly dissolved form.

The transesterification may be performed within a temperature range of 0-200° C. This range includes all values and subvalues therebetween, preferably at 10-100° C. and more preferably at 20-80° C.

The transesterification may be performed within a pressure range of 0.1-100 bar. This range includes all values and subvalues therebetween, preferably at 0.5-50 bar and most preferably at 1-5 bar.

The catalyst system may be mixed with the mono-, di- or triglyceride and optionally additional monohydric alcohol, the monohydric alcohol being consumed and glycerol released. It is essential that the entire catalyst system, i.e. the transesterification catalyst and the activator, is present as a mixture at the start of the transesterification reaction. The reaction catalyst used and the activator become, for the most part, distributed in the heavier glycerol phase which forms.

The reaction mixture may be worked up in different ways. Once the transesterification has been conducted to the desired conversion, preferably to 98% or higher, a fatty acid alkyl ester phase and a glycerol phase generally form, which may be separated readily by the person skilled in the art by known process steps, for example decanting. The inventive catalyst system accelerates the separation of the phases, which significantly eases the workup and increases the space-time yield.

The invention further provides for the use of the fatty acid alkyl esters obtainable by the process as a constituent of biodiesel (for example according to specification DIN EN 14214).

Even without further details, it is assumed that a person skilled in the art can utilize the above description in the widest scope. The preferred embodiments and examples should therefore be interpreted merely as descriptive disclosure which does not impose any kind of limit. The present invention is illustrated hereinafter with reference to examples. Alternative embodiments of the present invention are obtainable in an analogous manner.

Having generally described this invention, a further understanding can be obtained by reference to certain specific examples which are provided herein for purposes of illustration only, and are not intended to be limiting unless otherwise specified.

### EXAMPLES

### Example 1 and 1b

### noninventive

Duplicate experiments wherein 500 g of algae oil (approx. 0.57 mol), 58 g of methanol (1.81 mol) and 7 g of a 30% methanolic solution of sodium methoxide (0.04 mol of transesterification catalyst) were heated to 60° C., mixed and stirred for one hour, were conducted. Subsequently, the mixture was introduced into a separating funnel and the time taken for a clear lower glycerol phase to occur was measured. In the experiments described, this took 8:15 min and 8:54 min, respectively.

### Examples 2-20

### Inventive

500 g of algae oil (approx. 0.57 mol), 58 g of methanol (1.81 mol) and 7 g of a 30% methanolic solution of sodium methoxide (0.04 mol of transesterification catalyst) which contains various activators were heated to 60° C., mixed and stirred for one hour. Subsequently, the mixture was introduced into a separating funnel and the time taken for a clear lower glycerol phase to occur was measured. The results are listed in table 1.

TABLE 1

| | | Concen- | Time for phase |
|---|---|---|---|
| | | tration | separation |
| Ex. | Activator | [%] | [min:sec] |
| 1 | — | | 8:45 |
| 1b | — | | 8:15 |
| 2 | potassium chloride (KCl) | 15 | 4:30 |
| 3 | potassium chloride (KCl) | 30 | 5:00 |
| 4 | potassium methoxide | 11 | 5:00 |
| | (KOMe) | | |
| 5 | potassium methoxide | 17 | 4:00 |
| | (KOMe) | | |
| 6 | potassium ethoxide (KOEt) | 12 | 6:00 |
| 7 | potassium t-butoxide (KOt- | 12 | 5:00 |
| | Bu) | | |
| 8 | potassium nitrate | 12 | 5:30 |
| 9 | potassium carbonate (K₂CO₃) | 12 | 6:30 |
| 10 | rubidium chloride (RbCl) | 12 | 6:00 |
| 11 | potassium acetate (KOAc) | 12 | 6:30 |
| 12 | potassium formate (KO₂CH) | 12 | 4:30 |
| 13 | K₃[Fe(CN)₆] | 12 | 3:30 |
| 14 | K₄[Fe(CN)₆] | 12 | 5:30 |
| 15 | caesium chloride (CsCl) | 12 | 4:30 |
| 15 | potassium phosphate | 12 | 4:30 |
| | (K₃PO₄) | | |
| 16 | potassium thiocyanate | 12 | 5:00 |
| | (KSCN) | | |
| 17 | ethylene glycol | 12 | 4:00 |
| 18 | dimethylformamide | 12 | 3:30 |
| 19 | propionamide | 12 | 4:00 |
| 20 | glycerol | 30 | 4:10 |

### Example 21

### Noninventive

500 g of rapeseed oil (approx. 0.57 mol), 58 g of methanol (1.81 mol) and 8.5 g of a 32% methanolic solution of potassium methoxide (0.04 mol of transesterification catalyst) were heated to 60° C., mixed and stirred for one hour. Subsequently, the mixture was introduced into a separating funnel and the time taken for a clear lower glycerol phase to occur was measured. In the experiment described, this took 8 min.

### Examples 22-24

### Inventive

500 g of rapeseed oil (approx. 0.57 mol), 58 g of methanol (1.81 mol) and 8.5 g of a 32% methanolic solution of potassium methoxide (0.04 mol of transesterification catalyst) which contains various activators were heated to 60° C., mixed and stirred for one hour. Subsequently, the mixture was introduced into a separating funnel and the time taken for a clear lower glycerol phase to occur was measured.

The results are listed in table 2.

TABLE 2

| | | Concen- | Time for phase |
|---|---|---|---|
| | | tration | separation |
| Ex. | Activator | [%] | [min:sec] |
| 21 | — | | 8:00 |
| 22 | rubidium chloride (RbCl) | 11 | 3:30 |
| 23 | potassium chloride (KCl) | 11 | 4:00 |
| 24 | ethylene glycol | 11 | 4:00 |

### Example 25

### Noninventive

300 g of rapeseed oil (approx. 0.34 mol) and 35 g of methanol (1.09 mol) which contains 0.94 g (0.02 mol) of NaOH were heated to 60° C., mixed and stirred for one hour. Subsequently, the mixture was introduced into a separating funnel and the time taken for a clear lower glycerol phase to occur was measured. In the experiment described, this took 18 minutes.

### Example 26

### Iinventive

300 g of rapeseed oil (approx. 0.34 mol), 35 g of methanol (1.09 mol) which contains 0.94 g (0.02 mol) of NaOH and 0.6 g of a 32% methanolic potassium methoxide solution were heated to 60° C., mixed and stirred for one hour. Subsequently, the mixture was introduced into a separating funnel and the time taken for a clear lower glycerol phase to occur was measured. In the experiment described, this took 16 minutes.

### Example 27

### Noninventive

300 g of rapeseed oil (approx. 0.34 mol) and 35 g of methanol (1.09 mol) which contains 1.50 g (0.02 mol) of KOH are heated to 60° C., mixed and stirred for one hour. Subsequently, the mixture was introduced into a separating funnel and the time taken for a clear lower glycerol phase to occur was measured. In the experiment described, this took 21 minutes.

### Example 28

### Inventive

300 g of rapeseed oil (approx. 0.34 mol) and 35 g of methanol (1.09 mol) which contains 1.50 g (0.02 mol) of KOH and 0.6 g of rubidium chloride as an activator were heated to 60° C., mixed and stirred for one hour. Subsequently, the mixture was introduced into a separating funnel and the time taken for a clear lower glycerol phase to occur was measured. In the experiment described, this took 19 minutes.

In all examples, a distinct shortening of the time until phase separation was observed when a catalyst system for use in accordance with the present invention was used. Numerous modifications and variations on the present invention are possible in light of the above teachings. It is therefore understood that within the scope of the appended claims, the invention may be practiced otherwise than as specifically described herein.

1. A method for preparation of a fatty acid alkyl ester, comprising:
transesterifying at least one of a mono-, di- or triglyceride with a monohydric alcohol in the presence of a catalyst system to obtain a phase separated product mixture comprising a fatty acid alkyl ester phase and a glycerol phase;
wherein the catalyst system comprises:
a transesterification catalyst selected from the group consisting of an alkali metal, an alkaline earth metal alkoxide and an alkali metal hydroxide; and
at least one activator, different from the transesterification catalyst, selected from the group consisting of a salt compound, a titanate and a non-salt compound having a density of at least 0.9 g/ml.

2. The method according to claim 1, wherein the transesterification catalyst is sodium methoxide, potassium methoxide, sodium ethoxide, potassium ethoxide, sodium hydroxide or potassium hydroxide.

3. The method according to claim 1, wherein the at least one activator is a salt compound selected from the group consisting of a chloride, a bromide, a fluoride, an acetate, a formate, a phosphate, a hydrogenphosphate, a sulphate, a hydrogensulphate, a nitrate, a carbonate, a hydrogencarbonate, a cyanide, a cyanate, a thiocyanate, a borate, a silicate, an aluminate, an alkoxide and a hexacyanoferrate.

4. The method according to claim 1, wherein the at least one activator is a titanate selected from the group consisting of tetramethyl titanate, tetraethyl titanate and tetraisopropyl titanate.

5. The method according to claim 1, wherein the at least one activator is a non-salt compound having a density of at least 0.9 g/ml selected from the group consisting of ethylene glycol, diethylene glycol, formamide, dimethylformamide, N-methylformamide, acetamide, dimethylacetamide, N-methylacetamide, N-ethylacetamide, propanamide, N-methylpropanamide, N-ethylpropanamide, N-methylpyrrolidone and dimethyl sulphoxide.

6. The method according to claim 1, wherein a the concentration of the transesterification catalyst is 0.001-20% by weight, based on the amount of the mono-, di- or triglyceride.

7. The method according to claim 1, wherein a concentration of the activator is 0.01-25% by weight, based on an amount of the transesterification catalyst.

8. The method according to claim 1, wherein a temperature of the transesterification is from 0 to 200° C.

9. The method according to claim 1, wherein a pressure of the transesterification is 0.1-100 bar.

10. The method according to claim 1 , wherein the monohydric alcohol is selected from the group consisting of methanol, ethanol, n-propanol, isopropanol, n-butanol, isobutanol, sec-butanol or tert-butanol, amyl alcohol, tert-amyl alcohol, n-hexanol and 2-ethylhexanol.

11. The method according to claim 1, further comprising, after the transesterification:
separating the glycerol phase from the fatty acid alkyl ester phase.

12. The method according to claim 1, wherein a molar ratio of the monohydric alcohol to the least one a mono-, di- or triglyceride is from 3/1 to 20/1.

13. The method according to claim 1, further comprising:
mixing the catalyst system with the least one of a mono-, di- or triglyceride and monohydric alcohol prior to the transesterification.

14. The method according to claim 1, wherein the least one of a mono-, di- or triglyceride is a natural fat selected from the group consisting of olive oil, coconut fat, palm kernel fat, babassu oil, palm oil, palm kernel oil, peanut oil, rapeseed oil, corza oil, castor oil, sesame oil, sunflower oil, soya oil, hemp oil, poppy oil, avocado oil, cottonseed oil, wheatgerm oil, maize kernel oil, pumpkinseed oil, tobacco oil, grapeseed oil, jatropha oil, algae oil, karanja oil, oil of Pongamia pinnata, camelina oil, linseed dotter oil, cocoabutter, plant tallows, bovine tallow, pork fat, chicken fat, bone fat, mutton tallow, japan tallow, whale oil, a fish oil, and a train oil.

15. A method for preparing a biodiesel, comprising:
transesterifying at least one natural fat with a monohydric alcohol in the presence of a catalyst system to obtain a phase separated product mixture comprising a biodiesel phase and a glycerol phase; and
separating the glycerol phase from the biodiesel phase to obtain the biodiesel;
wherein the catalyst system comprises:
a transesterification catalyst selected from the group consisting of an alkali metal, an alkaline earth metal alkoxide and an alkali metal hydroxide; and
at least one activator, different from the transesterification catalyst, selected from the group consisting of a salt compound, a titanate and a non-salt compound having a density of at least 0.9 g/ml.

16. The method according to claim 15, further comprising:
mixing the catalyst system with the at least one natural fat and monohydric alcohol prior to the transesterification.

17. The method according to claim 15, wherein the monohydric alcohol is methanol and the transesterification catalyst is sodium methoxide.

18. The method according to claim 15, wherein the at least one activator is a salt compound selected from the group consisting of a chloride, a bromide, a fluoride, an acetate, a formate, a phosphate, a hydrogenphosphate, a sulphate, a hydrogensulphate, a nitrate, a carbonate, a hydrogencarbonate, a cyanide, a cyanate, a thiocyanate, a borate, a silicate, an aluminate, an alkoxide and a hexacyanoferrate.

19. The method according to claim 15, wherein the at least one activator is a titanate selected from the group consisting of tetramethyl titanate, tetraethyl titanate and tetraisopropyl titanate.

20. The method according to claim 15, wherein the at least one activator is a non-salt compound having a density of at least 0.9 g/ml selected from the group consisting of ethylene glycol, diethylene glycol, formamide, dimethylformamide, N-methylformamide, acetamide, dimethylacetamide, N-methylacetamide, N-ethylacetamide, propanamide, N-methylpropanamide, N-ethylpropanamide, N-methylpyrrolidone and dimethyl sulphoxide.

21. The method according to claim 15, wherein a content of triglycerides according to DIN EN 14214, is 0.2% by weight or less.

22. A biodiesel composition, comprising a biodiesel obtained by the method according to claim 15.

23. A biodiesel composition, comprising a biodiesel obtained by the method of claim 21.
